(19) United States
(12) Patent Application Publication
(10) Pub. No.: US 2017/0079855 A1
(43) Pub. Date: Mar. 23, 2017

(54) Colored-Back Disposable Pantiliner (Panty Liner) and Colored-Back Disposable Ultra-Thin Sanitary Napkin/Pad for Lace and Sheer Panties

(71) Applicants: Kellie Lanor Young, Detroit, MI (US); Kim Baneia Bradley, Detroit, MI (US)

(72) Inventors: Kellie Lanor Young, Detroit, MI (US); Kim Baneia Bradley, Detroit, MI (US)

(21) Appl. No.: 15/253,892

(22) Filed: Sep. 1, 2016

Related U.S. Application Data
(60) Provisional application No. 62/283,437, filed on Sep. 1, 2015.

(51) Int. Cl. A61F 13/514; A61F 13/15; A61F 13/531; A61F 13/472

(52) U.S. Cl. CPC A61F 13/51496; A61F 13/472; A61F 13/15203; A61F 13/531; A61F 2013/4708

(57) ABSTRACT

A disposable pantiliner (panty liner) or disposable ultra-thin sanitary napkin/pad characterized by a colored nonwhite, nonpink “nonwoven backing” located on the bottom or last layer of the pantiliner or ultra-thin sanitary napkin/pad. This colored nonwoven backing is the part or section of the pantiliner that has an adhesive glue strip that is pressed against the crotch region of the sheer or lace panty or other undergarment. This colored nonwoven backing is also plastic and the section or part that will “blend in” with “matching in color” with the sheer or lace panty or other undergarment worn, becoming invisible or unnoticeable.

### CROSS-REFERENCE TO RELATED APPLICATION

This Non-Provisional Utility application claims the benefit of U.S. Provisional Application No. 62/283,437 filed, Sep. 1, 2015.

### FIELD OF THE INVENTION

The present invention relates to disposable feminine hygiene articles, in particular pantiliners (panty liners) and ultra-thin sanitary napkins/pads. More specifically, the invention relates to an improved pantiliner and ultra-thin sanitary napkin's/pad's bottom back layer.

### BACKGROUND OF THE INVENTION

Disposable pantiliners (panty liners) and ultra-thin sanitary napkins/pads; feminine hygiene articles, have long been known and widely used by women to protect the undergarment or panty from soling due to vaginal discharge during ovulation, blood from menstrual flow or perspiration (sweat).

The disposable pantiliner and ultra-thin sanitary napkin/pad consisting of three layers: a top layer, a thin absorbent cotton layer (which also includes a plastic fluid impermeable barrier), and a bottom back layer. The top layer allows moisture to be pulled back away from the skin into the absorbent cotton, where the plastic fluid impermeable barrier traps the liquid or fluid. The back layer which is positioned on the bottom faces away from the body and has an adhesive surface whereby the pantiliner can be attached and secured to the inner crotch region of the undergarment preventing slippage.

The term “back layer” is commonly used in the industry or trade, and synonymous with other terms commonly called, used or referred to as “backsheet,” “bottomsheet,” “bottom layer,” “outer layer,” “last layer,” “backing,” “plastic backing,” “plastic back layer” and “bottom back layer.”

The back layer of the disposable pantiliner (panty liner) or disposable ultra-thin sanitary napkin/pad, which is in contact with the panty, underwear or undergarment, and providing for the adhesive attachment directly to the crotch region of the undergarment; has a common designed color of “pink” or “white.” Typical pantiliners of this type are shown, for example, in the following United States of America patents:

Patent No. WO2004039298 A1 issued to Zimmerman on May 13, 2004 discloses a conventional pantiliner may be improved such that the above may be matched to the large number of possible colors of underwear and also an absorption of colors through the skin is avoided, whereby merely the outer layer of the panty liner, which is in contact with the underwear, has a color which is not white.

U.S. Pat. No. 4,548,603 issued to Ichijo on Oct. 22, 1985 discloses a sanitary napkin where the outer surface is colored with an image pattern.

Patent No. DE29824115U1 issued to Manfred on Jun. 21, 2000 discloses an absorbent product for excreta of the human body such as a panty liner or a sanitary napkin, characterized by an absorbent body having an intended for contact with a wearer's body inner side and an intended for contact with a garment of the wearer outside, that at least the outside of a particular color a dye, a print, such as a pattern and/or a text, and/or having a material support.

However, the pantiliner and ultra-thin sanitary napkin/pad bottom back layer's common color design has a severe inconvenience of being highly visible and seen through lace or sheer panties or other undergarment being worn.

Although pantiliners have achieved considerable fame and commercial success, there has been a continuing need for improvement.

While various disposable pantiliners (panty liners) and ultra-thin sanitary napkins/pads for female hygiene exist, none relate to the design and function of improving women self-esteem and confidence in keeping the sexy look of their lace and sheer panties or other undergarments when wearing a pantiliner according to the present invention.

### BRIEF SUMMARY OF THE INVENTION

One of the things that makes the present invention unique is the “objective” of the design and function of the disposable pantiliner's colored-back bottom layer. The present invention is produced in a variety of colors. The colored back consists of one solid color (other than pink or white) for its backing. The woman will select the colored-back disposable pantiliner that matches the color lace or color sheer panty being worn. Remove the adhesive wrapper from the colored-back disposable pantiliner, position, place and press the colored-back (sticky/bottom part) in the crotch of the matching color panty or other undergarment. Once the colored-back disposable pantiliner or colored-back disposable ultra-thin sanitary napkin/pad is placed in the matching colored panty being worn, the colored-back layer (which has the sticky/bottom part) of the pantiliner will “blend in” with the color panty becoming “invisible” or “unnoticeable.” This invention provides a sexier appeal and look, as well as eliminating the unnoticeable, unsightly common colored “pink” or “white” pantiliner appearance shown through the panty or undergarment not white or pink in color.

### BRIEF DESCRIPTION OF THE DRAWINGS

A clear understanding of the key features of the invention summarized above may be had by reference to the appended drawings, which illustrates the design and function of the invention, although it will be understood that such drawings depict preferred embodiments of the invention and, therefore, are not to be considered as limiting its scope with regard to other embodiments which the invention is capable of contemplating. Accordingly:

FIG. 1A-FIG. 1B are illustrations of the design and function of the invention showing a simplified end-view diagram of a disposable pantiliner or disposable ultra-thin sanitary napkin/pad; a feminine hygiene article that has been created with the white or pink plastic bottom layer facing the inside crotch area of a non-white, non-pink colored lace or non-white, non-pink colored sheer panty.

FIG. 2A-FIG. 2D are illustrations of the design and function of the invention showing a simplified end-view diagram of a colored-back disposable pantiliner (panty liner) or colored-back disposable ultra-thin sanitary napkin/pad for lace and sheer panties; a feminine hygiene article that has been created with a colored non-white, non-pink bottom layer facing the inside crotch area of a colored non-white, non-pink lace or colored non-white, non-pink sheer panty matching in color with the bottom layer of the pantiliner (panty liner).

FIG. 3A is a bottom-view diagram illustrating the preferred embodiment of the design and function of the invention using the preferred colored-back disposable pantiliner (FIG. 3C) that has been positioned in the crotch area of the lace panty of the same color (FIG. 3B).

FIG. 4 is a bottom-view illustration diagram of the preferred embodiment of the invention illustrated in FIG. 3 showing the colored-back disposable pantiliner or colored-back disposable ultra-thin sanitary napkin/pad matching in color with the lace or sheer panty or other undergarment worn.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It is also understood that the term “whereby” is used herein to introduce a function or result at the end of a claim, and the term “wherein” is used herein in a dependent claim to recite an element or part more specifically. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical, scientific, trade or industry terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques, steps, color spectrums, fabrics and ethnicities or nationalities are disclosed. Each of these has individual benefit and each can also be used in conjunction with one or more, or in some cases all, of the other disclosed techniques and/or color spectrums. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

New and improved colored-back disposable pantiliner and colored-back disposable ultra-thin sanitary napkin/pad plastic bottom layer or back layer's designs and functions are discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention, and is not intended to limit the invention to the specific embodiments illustrated by the figures or description below.

The present invention will now be described by referencing the appended figures representing preferred embodiments.

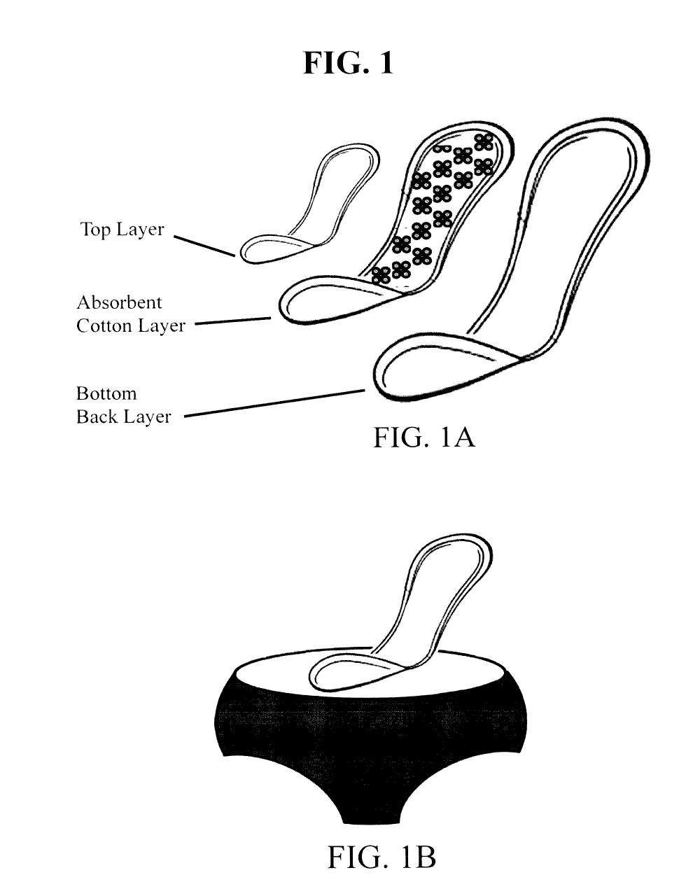

FIG. 1A depicts an exploded perspective view of the elements that may comprise a disposable pantiliner (panty liner) or a disposable ultra-thin sanitary napkin/pad with a common designed plastic back layer or plastic bottom layer, white or pink in color, facing the inside crotch area (FIG. 1B) of a non-white, non-pink colored lace or sheer panty indicating the contrast in color between the pantiliner and the panty.

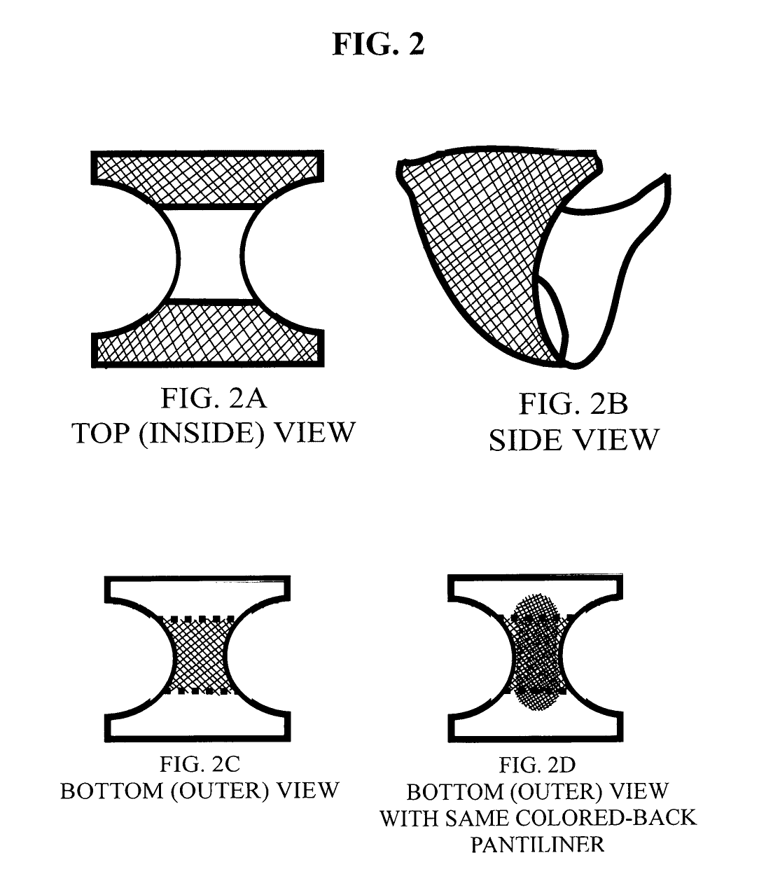

FIG. 2A-FIG. 2D depicts an orthographic projection drawing of the top (inside) view, side (inside) view and bottom (outer) view of the panty with the pantiliner's or ultra-thin sanitary napkin/pad's plastic bottom layer facing away from the body inside of the lace or sheer panty's crotch area, visibly matching the bottom (outer) panty in color.

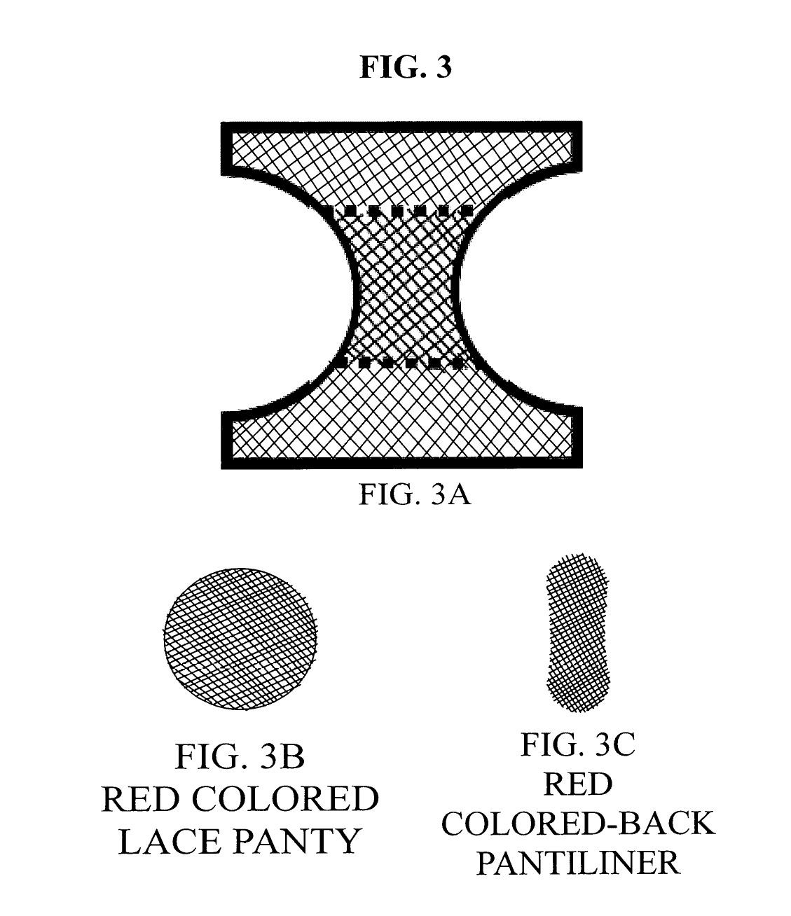

FIG. 3C illustrates the disposable colored-back pantiliner that has been positioned in the lace panty of the same color (FIG. 3B) whereby no unwanted negative attention is drawn to the crotch area.

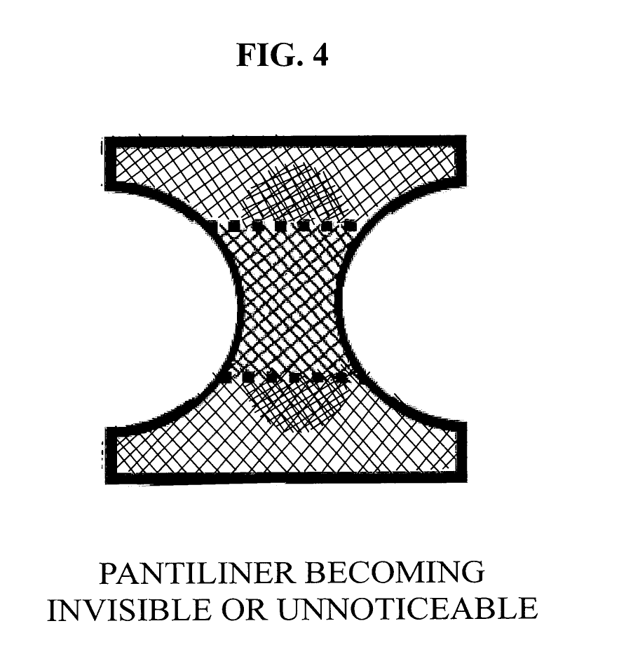

FIG. 4 further illustrates the disposable colored-back pantiliner's plastic bottom layer pressed against the lace panty of the same color, becoming “invisible” or “unnoticeable.”

In preferred embodiments, the disposable solid plastic colored-back layer is used and placed as the bottom layer of the disposable pantiliner (panty liner) or disposable ultra-thin sanitary napkin/pad. The embodiment allows lace and sheer colored panties that are other than white or pink in color to keep their sexy look and appeal without the unsightly pantiliner showing through when worn with panties or other undergarments that matches the colored-back bottom layer of the pantiliner. Another important feature of this design and function is that the woman's confidence and self-esteem continues to stay elevated because the colored-back disposable pantiliner is not negatively drawing attention whereby it's seen by her partner before or during intimacy.

The present invention allows the disposable colored-back pantiliners (panty liners) or disposable ultra-thin sanitary napkins/pads to be produced in a color spectrum range of various solid colors, from pastels to neon colors to solid bright colors for lace and sheer panties, as well as being produced in an ethnic color spectrum range of various flesh-tone and skin-tone colors that gives a more “natural” skin color appearance through the lace or sheer panties or other undergarment worn.

While the present invention has been described in terms of particular embodiments and applications, in both summarized and detailed forms, it is not intended that these descriptions in any way limit its scope to any such embodiments and applications, and it will be understood that many substitutions, changes and variations in the described embodiments, applications and details of the design and function illustrated herein and of their operation can be made by those skilled in the art without departing from the spirit of this invention.

### PATENT CITATIONS

| Cited Patent | Filing date | Publication date | Applicant | Title |
|---|---|---|---|---|
| WO2004039298 A1 | Oct. 20, 2003 | May 13, 2004 | Fred Zimmerman | Panty liner and method |
| U.S. Pat No. 4,548,603 A | Jul. 25, 1983 | Oct. 22, 1985 | Teruko Ichijo | Sanitary Napkin |
| DE29824115U1 | Nov. 3, 1998 | Jun. 21, 2000 | Kreupl Manfred | The absorbent product |
| WO2005/084597 | Mar. 1, 2005 | Sep. 15, 2005 | Paolo Veglio (+4) | Pantiliner |
| WO2009007882 A1 | Jul. 2, 2008 | Jan. 15, 2009 | Roberto D'addario | Pantiliner |
| WO2002007665 A1 | Jul. 9, 2001 | Jan. 31, 2002 | Helene Karin Costea (+3) | Package for . . . |
| EP1174101 A1 | Jul. 21, 2000 | Jan. 23, 2002 | Giovanni Carlucci (+4) | Dark colored . . . |
| CA2469007C | Nov. 27, 2002 | Oct. 7,2008 | Lilkar Z. Molina (+1) | Package . . . |
| U.S. Pat. No. 2,929,379 | Jul. 29, 1957 | Mar. 22, 1960 | Poulsen Else Vita | Sanitary Napkin |
| U.S. Pat. No. 5,429,631 | Jul. 30, 1993 | Jul. 4, 1995 | Dyandra Grenier | Sanitary article |
| US20080097364 A1 | Oct. 22, 2006 | Apr. 24, 2008 | Yinhai Yang | Method of . . . |
| WO2003013406 A1 | Aug. 2, 2002 | Feb. 20, 2003 | Herman P. Benecke (+5) | Color masking |
| WO2016014101 A1 | Oct. 15, 2014 | Jan. 28, 2016 | Lauren Giordano (+1) | Absorbent . . . |
| U.S. Pat. No. 9,173,783 B1 | Oct. 3, 2014 | Nov. 3, 2015 | Juliana Terian (+1) | Absorbent . . . |
| WO2008146222 A1 | May 23, 2008 | Dec. 4, 2008 | Cornelia Ecker (+2) | Feminine . . . |
| US20150080824 A1 | Nov. 21, 2014 | Mar. 19, 2015 | Cornelia Ecker (+2) | Feminine . . . |
| U.S. Pat. No. 7,388,123 B2 | Dec. 30, 2002 | Jun. 17, 2008 | Christine M. Cowell (+2) | Feminine care |
| WO2011056689 A1 | Oct. 28, 2010 | May 12, 2011 | Kelyn Ann Arora (+1) | Absorbent . . . |
| U.S. Pat. No. 8,629,315 B2 | May 6, 2011 | Jan. 14, 2014 | Tsunetoshi Miura (+5) | Absorbent . . . |
| USD745666 S1 | Nov. 13, 2013 | Dec. 15, 2015 | Jeannine Cardin (+3) | Sanitary napkin |
| WO2011015956 A2 | Jun. 30, 2010 | Feb. 10, 2011 | Heidi Hopkins (+3) | Absorbent . . . |
| US20030114811 A1 | Dec. 19, 2001 | Jun. 19, 2003 | Patricia Christon (+3) | Absorbent . . . |
| U.S. Pat. No. 7,241,280 B2 | Sep. 20, 2004 | Jul. 10, 2007 | Patricia Christon (+3) | Absorbent . . . |
| US20120165769 A1 | Sep. 11, 2007 | Jun. 28, 2012 | Gabriela Roche del Ayala | Sanitary napkin |
| WO2011056777 A1 | Nov. 2, 2010 | May 12, 2011 | Kelyn Ann Arora (+1) | Method of . . . |
| | | | . . . | |
| U.S. Pat. No. 8,052,664 B2 | — | Nov. 8, 2011 | Youshiyuki Shimizu (+3) | Packaged ab . . . . |

What is claimed is:

1) A disposable pantiliner or disposable ultra-thin sanitary napkin/pad comprising: a) a nonwoven top sheet, layer or cover b) a middle layer or absorbent core, including a fluid impermeable plastic layer and c) a colored back, bottom layer or backing facing away from the body towards the garment.

2) A pantiliner or ultra-thin sanitary napkin/pad of claim 1, further comprising: back layer of a solid color other than the common white or pink in color.

3) A pantiliner or ultra-thin sanitary napkin/pad of claim 2, wherein said back layer preventing fluids from soaking through to panty or other undergarment has a solid color spectrum range consisting of any or all colors in the rainbow.

4) A pantiliner or ultra-thin sanitary napkin/pad of claim 3, wherein said back layer has a color spectrum range from light pastels to neon colors to bright solid colors to dark solid colors.

5) A pantiliner or ultra-thin sanitary napkin/pad of claim 4, wherein said back layer consisting of one solid color blending with or matching in color with lace or sheer panties or other undergarment being worn.

6) A pantiliner or ultra-thin sanitary napkin/pad of claim 5, wherein said back layer consisting of same matching color of lace or sheer panties or other undergarment worn whereby the pantiliner or ultra-thin sanitary napkin/pad becomes “invisible” or “unnoticeable” through the panties or other undergarment worn.

7) A disposable pantiliner or disposable ultra-thin sanitary napkin/pad comprising: a back layer of solid non-white, non-pink color wherein the back layer is made of a pigmented nonwoven material facing away from the body.

8) A pantiliner or ultra-thin sanitary napkin/pad of claim 7, further comprising: back layer of a solid color “natural” in appearance matching the “flesh-tone” or “skin-tone” of the woman's own skin color seen through the undergarment worn.

9) A pantiliner or ultra-thin sanitary napkin/pad of claim 8, wherein said back layer has a solid color spectrum range consisting of any and all colors of women of all ethnicity or nationalities.

10) A pantiliner or ultra-thin sanitary napkin/pad of claim 9, wherein said back layer has color spectrum ranges for women who are white/Caucasian, black/African, Asian, Hispanic, Native American and etc.

11) A pantiliner or ultra-thin sanitary napkin/pad of claim 10, wherein said back layer consisting of one solid color “naturally” matching the skin-tone color of any woman of ethnicity and “unnoticeable” when seen through the panty or other undergarment worn.
